(19) United States
(12) Patent Application Publication
(10) Pub. No.: US 2023/0091740 A1
(43) Pub. Date: Mar. 23, 2023

(54) STENT GRAFTS HAVING A RADIOPAQUE MARKER AND METHODS OF PRODUCING

(71) Applicant: C.R. Bard, Inc., Franklin Lakes, NJ (US)

(72) Inventors: Robert Burgmeier, Plymouth, MN (US); Cyal Lecy, Buffalo, MN (US)

(21) Appl. No.: 17/904,516

(22) PCT Filed: Feb. 26, 2020
(86) PCT No.: PCT/US2020/019827

§ 371 Date: Aug. 18, 2022

(51) Int. Cl. A61F 2/07; A61L 31/18; A61L 31/14; A61L 31/04; C08L 27/18; C08L 65/04

(52) U.S. Cl. CPC A61F 2/07; A61L 31/18; A61L 31/146; A61L 31/048; C08L 27/18; C08L 65/04

(57) ABSTRACT

In an embodiment, a method of producing a radiopaque stent graft is provided. The method may include functionalizing a surface of a coating layer of a stent graft to produce a functionalized surface; disposing a tie layer over the functionalized exterior surface of the stent graft; disposing an adherent layer over the tie layer; etching a slot into the adherent layer; functionalizing the slot; and positioning a radiopaque marker in the slot to produce a filled slot and thereby produce the radiopaque stent graft.

### TECHNICAL FIELD

The present specification generally relates to medical devices and, more specifically, stent grafts that include radiopaque markers.

### BACKGROUND

In the medical field, implantable prostheses are used to support vessels in a mammalian body, such as vessels in the urethra, esophagus, biliary tract, intestines, arteries, veins, and peripheral vessels. For example, implantable prostheses may be utilized to support a vessel in the mammalian body that suffers from an abnormal widening (e.g., an aneurysm, vessel contraction or lesion such as a stenosis or occlusion) or an abnormal narrowing (e.g., a stricture). In some instances, stents may be utilized. Such prostheses may include frame-like structures, such as stents, stent grafts, and covered stents.

However, conventional stents, stent grafts, and covered stents may lack the ability to be seen clearly under fluoroscopy, which can hamper the placement of the implantable prostheses. Accordingly, there are needs for radiopaque stents, stent grafts, and covered stents and methods of making radiopaque stents, stent grafts, and covered stents.

### SUMMARY

Embodiments of the present disclosure meet these needs by providing radiopaque stents, stent grafts, and covered stents and methods of making radiopaque stents, stent grafts, and covered stents.

According to one aspect of the present disclosure, a method of producing a radiopaque stent graft is provided. The method may include treating an exterior surface of a base stent graft to produce a functionalized surface; disposing a tie layer over the functionalized exterior surface; disposing an adherent layer over the tie layer; etching a slot into the adherent layer to produce an etched layer; treating the etched layer; and positioning a radiopaque marker in the slot to produce a filled slot and thereby produce the radiopaque stent graft.

A second aspect may include the first aspect, wherein the base stent graft comprises polytetrafluoroethylene.

A third aspect may include any of the preceding aspects, wherein the adherent layer comprises a poly(p-xylylene).

A fourth aspect may include the third aspect, wherein the poly(p-xylylene) comprises one or more of parylene C, parylene N, parylene D, parylene X, parylene AF-4, parylene SF, parylene HT, parylene VT-4 (parylene F), parylene CF, parylene A, or parylene AM.

A fifth aspect may include any of the preceding aspects, further comprising coating the filled slot with an adhesive to produce a sealant layer.

A sixth aspect may include any of the preceding aspects, further comprising etching one or more additional slots in to the adherent layer; treating the one or more additional slots; positioning a radiopaque marker into each of the one or more additional slots to produce one or more additional filled slots and thereby produce the radiopaque stent graft.

A seventh aspect may include the sixth aspect, further comprising coating the filled slots with an adhesive to produce a sealant layer.

An eighth aspect may include any of the first or fifth aspects, wherein coating the one or more additional filled slots comprises utilizing a flexible acrylate adhesive.

A ninth aspect may include the eighth aspect, further comprising curing the adhesive to produce the sealant layer.

A tenth aspect may include any of the preceding aspects, wherein etching comprises laser-etching.

An eleventh aspect may include any of the preceding aspects, wherein treating the exterior surface comprises plasma-treating the exterior surface.

An twelfth aspect may include any of the preceding aspects, wherein treating the etched layer comprises plasma-treating the etched layer.

According to a thirteenth aspect of the present disclosure, a radiopaque stent graft is provided. Embodiments of the radiopaque stent graft may include a stent graft having a functionalized surface; a tie layer surrounding a functionalized surface; and an etched layer covering at least a portion of the tie layer, wherein the etched layer comprises a filled slot, and wherein the filled slot comprises a radiopaque marker.

A fourteenth aspect may include the thirteenth aspect, further comprising a sealant layer encapsulating at least a portion of the filled slot.

A fifteenth aspect may include any of the thirteenth through fourteenth aspects, wherein the sealant layer comprises a flexible acrylate adhesive.

A sixteenth aspect may include any of the thirteenth through fifteenth aspects, wherein the base stent graft comprises polytetrafluoroethylene.

A seventeenth aspect may include any of the thirteenth through sixteenth aspects, wherein the etched layer further comprises a poly(p-xylylene).

An eighteenth aspect may include the seventeenth aspect, wherein the poly(p-xylylene) comprises one or more of parylene C, parylene N, parylene D, parylene X, parylene AF-4, parylene SF, parylene HT, parylene VT-4 (parylene F), parylene CF, parylene A, or parylene AM.

A nineteenth aspect may include any of the thirteenth through eighteenth aspects, further comprising one or more additional filled slots, wherein the one or more additional filled slots each comprise a radiopaque marker.

A twentieth aspect may include any of the thirteenth through nineteenth aspects, wherein the radiopaque marker comprises a radiopaque material.

A twenty-first aspect may include any of the thirteenth through twentieth aspects, wherein the etched adherent layer is functionalized to securely affix the radiopaque marker to the slot.

A twenty-second aspect may include any of the thirteenth through twenty-first aspects, wherein the tie layer has a thickness of from 10 angstroms to 1,000 angstroms.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1A is an illustrative depiction of a side view of a radiopaque stent graft, according to one or more embodiments shown and described herein;

FIG. 1B is an illustrative depiction of a front view of a radiopaque stent graft, according to one or more embodiments shown and described herein;

FIG. 1C is an illustrative depiction of a side view of a radiopaque stent graft including an optional sealant layer, according to one or more embodiments shown and described herein;

FIG. 1D is an illustrative depiction of a front view of a radiopaque stent graft including an optional sealant layer, according to one or more embodiments shown and described herein;

FIG. 2 is an illustrative depiction of a side view of a base stent graft;

FIG. 3 is an illustrative depiction of a side view of a stent graft with a modified exterior surface;

FIG. 4 is an illustrative depiction of a side view of a stent graft with an adherent layer;

FIG. 5 is an illustrative depiction of a side view of a stent graft with an etched layer;

FIG. 6 is an illustrative depiction of a side view of a radiopaque stent graft with an etched adherent layer having filled slots, according to one or more embodiments shown and described herein;

FIG. 7 is an illustrative depiction of a side view of a radiopaque stent graft with an optional sealant layer, according to one or more embodiments shown and described herein.

### DETAILED DESCRIPTION

Embodiments as described herein are directed to radiopaque stent grafts and methods of producing a radiopaque stent grafts.

Implantable prostheses, such as stents, stent grafts, and covered stents, are currently used to treat narrowed (stenosed) tubular body vessels where the flow of blood or other biological fluids is restricted. For example, such vessels may be in the urethra, esophagus, biliary tract, intestines, arteries, veins, and peripheral vessels. Among other treatments, the implantable prostheses can be used to prop open blood vessels and reinforce collapsed or stenosed vessels in the respiratory system, reproductive system, bile or liver ducts or any other vessel.

In conventional treatment procedure methods, implantable prostheses may be implanted into the body by guiding a constraining member (i.e. a catheter) containing the implantable prosthesis to a treatment site within a body vessel. To locate the implantable prosthesis within the body vessel during deployment, the physician will generally utilize a fluoroscope, which uses X-rays, to visualize radiopaque markers on the implantable prosthesis or constraining member. However, if the radiopaque marker is located on the constraining member, and the constraining member is removed, correct placement of the implantable prosthesis cannot be confirmed. Accordingly, in order to implant the prosthesis by fluoroscopy, some portion of the implantable prosthesis should preferably be radiopaque.

However, incorporating a radiopaque marker directly into an implantable prosthesis may cause damage to the device. Under conventional methods, in order to incorporating a radiopaque marker into the device (i.e. by etching a slot in the surface of the device to receive the radiopaque marker), the modifications to the surface of the device may damage the properties of the device. For example, such conventional methods could cause the device to leak. One example includes stent grafts.

Stent grafts refer to stents used in combination with vascular grafts, which provide prostheses capable of maintaining their fit against blood vessel walls. Stent grafts may also be used in place of stents to prevent restenosis, which may occur where smooth muscle cells and other tissues can grow through a stent's mesh-like openings. Stent grafts may typically include expandable stents encapsulated within polytetrafluoroethylene (PTFE). PTFE is often used because it is biocompatible, light, porous and readily colonized by living cells. As used herein, “biocompatible” means causing little or no immunogenic reaction when placed within a mammalian body. Although there are advantages to using stent grafts comprising PTFE, the PTFE surface itself is not reactive. Accordingly, it may be nearly impossible to attach radiopaque markers to the PTFE surface of conventional stent grafts without damaging the stent graft.

Accordingly there are needs for radiopaque stent grafts and improved methods of making radiopaque stent grafts. Moreover there are needs for methods of making radiopaque stent grafts that may cause little or no damage to the stent graft. Embodiments of the radiopaque stent grafts described herein may be useful to confirm the correct placement of the stent within the vessel during a treatment procedure. Accurate visualization of the radiopaque stent grafts described herein may minimize the invasiveness of the procedure and allow for further visualization during follow-up inspections.

Radiopaque Stent Grafts

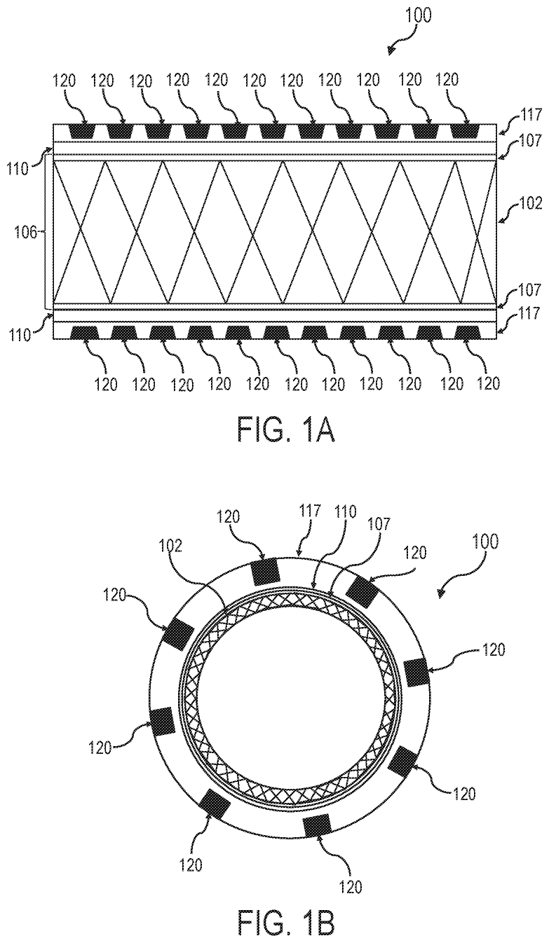

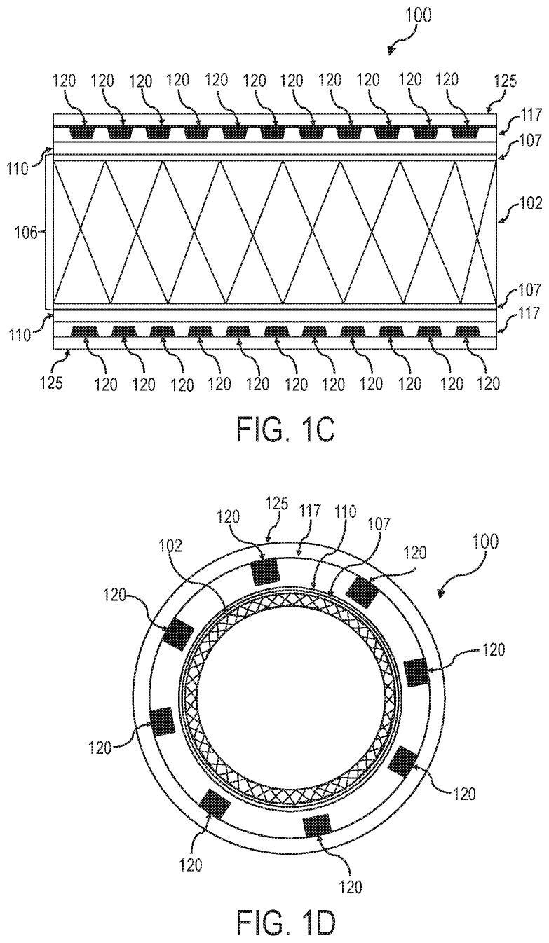

Embodiments of radiopaque stents grafts will now be described. FIG. 1A and FIG. 1B are illustrative depictions of a side view and front view, respectively, of a radiopaque stent graft 100, according to one or more embodiments shown and described herein. FIG. 1C and FIG. 1D are illustrative depictions of a side view and front view, respectively, of a radiopaque stent graft 100 with an optional sealant layer, according to one or more embodiments shown and described herein.

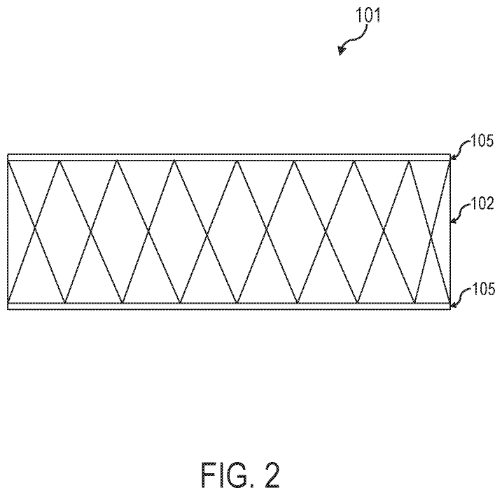

Referring to FIGS. 1A-1D and FIG. 2, in embodiments, the radiopaque stent graft 100 may include a stent graft 106 with a functionalized surface 107. The stent graft 106 may be produced from a base stent graft 101, such as the one shown in FIG. 2, having an expandable stent 102 encapsulated within a vascular graft 105.

Referring still to FIG. 2, the expandable stent 102 of the base stent graft 101 may be a balloon expanded or self-expanding stent. The expandable stent 102 may be made by various manufacturing methods, including forming wire and machining a hollow tube. Machining methods may include one or more of photo-chemical etching, laser-cutting, stamping, piercing, or other material-removal processes. Other manufacturing methods may include vacuum or chemical deposition of material or forming a tube of machined flat material.

In embodiments, the expandable stent 102 of the base stent graft 101 may be configured as a series of rings connected together to form a lattice-like framework thereby defining a tubular framework. In embodiments, the series of rings may or may not be essentially identical. In embodiments, the rings may be arranged in a circular configuration, a helical configuration, or a combination of helical and circular frameworks. In embodiments, the series of rings may or may not have connecting linkages between the adjacent rings. In embodiments that do not utilize connecting linkages between adjacent rings, the series of rings may be connected by a direct connection between one ring and the next ring. The series of rings may be placed adjacent to each other in order to maintain an appropriate longitudinal spacing between each rings.

The expandable stent 102 of the base stent graft 101 may comprise a biocompatible metal alloy. Examples of biocompatible metal alloys may include stainless steel, Nitinol or Elgiloy. In embodiments, the shape memory characteristics of Nitinol may allow the expandable stent 102 of the base stent graft 101 to self-expand when placed in a tubular body vessel at normal body temperature.

In embodiments, the vascular graft 105 of the base stent graft 101 may comprise PTFE, polyvinyl chloride (PVC), polyethylene terephthalate (PET), polyethylene, Nylon, PEBAX (i.e. a copolymer of polyether and polyamide), polystyrene (PS), polyethleneterephthalate (PETP), Dacron mesh reinforced umbilical tissues, bovine collagen, polyester knitted collagen, tricot knitted polyester collagen impregnated, polyurethane (available under the trade mark “Vectra R”) or various other suitable materials as will be apparent to those of ordinary skill in the art. In embodiments, the base stent graft 101 may be a self-expanding stent encapsulated within PTFE. In embodiments, the base stent graft 101 may be a self-expanding Nitinol stent encapsulated within PTFE. One example is known commercially as “Fluency®,” which is marketed by C.R. Bard Peripheral Vascular Inc.

In embodiments, the thickness of the vascular graft 105, the “wall thickness,” may provide flexibility to the base stent graft 101 to help assist manipulation of the radiopaque stent graft 100 during implantation. In embodiments, wall thickness of the vascular graft 105 may affect the overall size of the radiopaque stent graft 100 and the associated delivery system. For example, in embodiments, a relatively thinner wall thickness may allow for a relatively smaller radiopaque stent graft 100 to be manufactured, which may be delivered using a relatively smaller size catheter based delivery system. Additionally, wall thickness may affect the structural integrity of the radiopaque stent graft 100. For example, a thin wall thickness may be prone to structural degradation or kinking during implantation.

In embodiments, the wall thickness of the vascular graft 105 may range from about 50 micrometers (microns) to about 1000 microns. In embodiments, the wall thickness of the vascular graft 105 may range from about 50 microns to about 800 microns, from about 50 microns to about 600 microns, from about 50 microns to about 400 microns, from about 50 microns to about to about 200 microns, from about 50 microns to about 100 microns, from about 100 microns to about 1000 microns, from about 100 microns to about 800 microns, from about 100 microns to about 600 microns, from about 100 microns to about 400 microns, from about 100 microns to about to about 200 microns, from about 200 microns to about 1000 microns, from about 200 microns to about 800 microns, from about 200 microns to about 600 microns, from about 200 microns to about 400 microns, from about 400 microns to about 1000 microns, from about 400 microns to about 800 microns, from about 400 microns to about 600 microns, from about 600 microns to about 1000 microns, from about 600 microns to about 800 microns, or from about 800 microns to about 1000 microns.

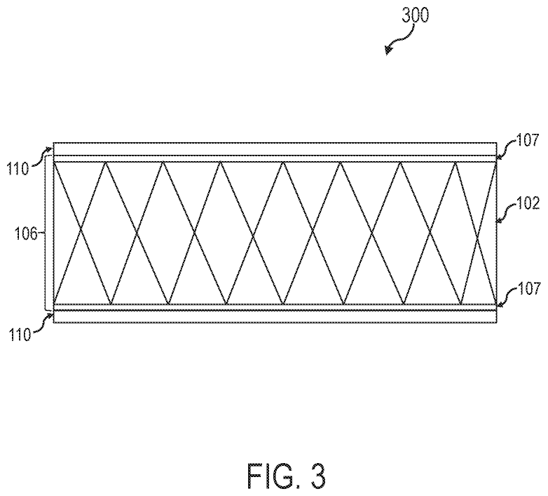

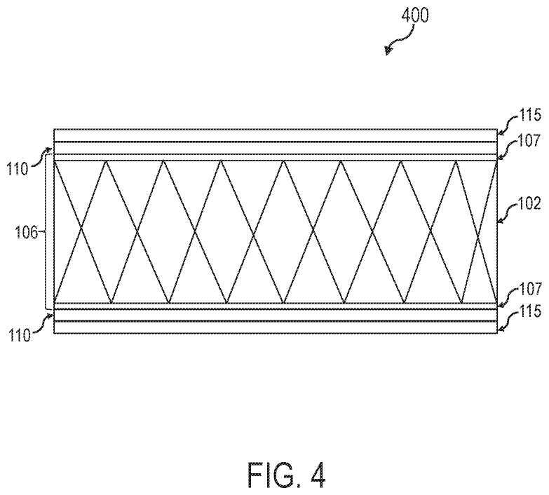

Referring now to FIGS. 3-4, the radiopaque stent graft 100 may include a functionalized surface 107, namely, a surface that has been subjected to a surface modification that decreases a surface free energy of the exterior surface of the vascular graft 105 before application of an adherent layer 115 (as shown in FIG. 4 and discussed in more detail below). As will be described below in more detail, the surface modification may include plasma treatment of the exterior surface of the vascular graft 105.

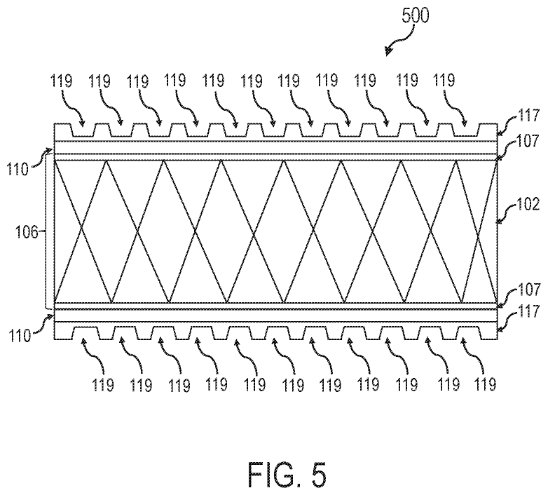

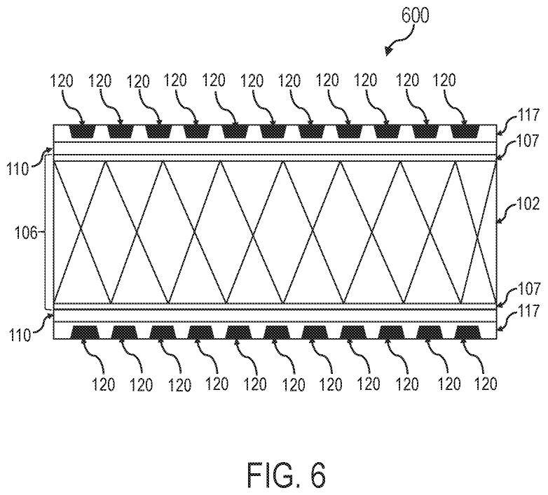

Referring now to FIGS. 5-6, in embodiments, the etched layer 117 may further include one or more slots 119, where “slots” may refer to depots or surface features formed by etching the adherent layer 115. Subsequently one or more radiopaque markers are applied to the one or more slots 119 thereby producing one or more filled slots 120. The radiopaque markers may fill at least a portion of the depots or surface features in the etched layer 117. Accordingly, the one or more filled slots 120 comprising one or more radiopaque markers allow for fluoroscopic visualization of the radiopaque stent graft 100 during implantation. In embodiments, the one or more radiopaque markers have radiopaque features incorporated therein, which can be used as visual markers located by fluoroscopy.

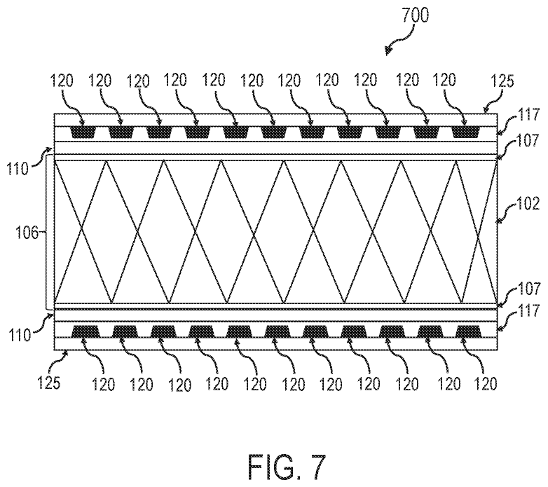

Referring now to FIG. 7, because the radiopaque stent graft 100 may be visualized by fluoroscopy during implementation, deployment of the presently-disclosed radiopaque stent grafts at unintended locations may be prevented. Additionally, the invasiveness associated with multiple deployment attempts or relocation may be reduced or eliminated. In embodiments, visualization of the radiopaque stent graft 100 may be utilized for both implantation and follow-up inspection during treatment.

In some embodiments, the radiopaque stent graft 100 may include an sealant layer 125, which includes an adhesive coated over the one or more filled slots 120.

Methods of Producing Radiopaque Stent Grafts

Embodiments of methods of producing radiopaque implantable prostheses will now be described. In embodiments, the disclosed methods may produce radiopaque stent grafts, as previously described in this disclosure. Although, the following method will be described as a method of producing a radiopaque stent graft, it should be understood that other implantable prostheses are also contemplated.

Referring now generally to FIGS. 2-7, embodiments of the method of producing a radiopaque stent graft 100 may include functionalizing the exterior surface of the vascular graft 105 of a base stent graft 101 to produce a stent graft 106 with a functionalized surface 107; disposing a tie layer 110 over the functionalized surface 107; disposing an adherent layer 115 over the tie layer 110; etching one or more slots 119 into the adherent layer 115; functionalizing the one or more slots 119; and positioning a radiopaque marker in the one or more slots 119 to produce one or more filled slots 120 and thereby produce the radiopaque stent graft 100.

As stated previously, FIG. 2 is an illustrative depiction of a side view of a base stent graft 101. In embodiments, the base stent graft 101 includes a vascular graft 105, which has an exterior surface. The exterior surface of the vascular graft 105 may be inert. In embodiments, the exterior surface of the vascular graft 105 of the base stent graft 101 may be subjected to an exemplary surface modification process to functionalize the exterior surface of the vascular graft 105 and thereby produce a stent graft 106 with a functionalized surface 107, as shown in FIG. 3. Referring to FIGS. 2-3, the surface modification may increase the functionality of the exterior surface of the vascular graft 105 producing a functionalized surface 107 that one or more layers may be applied to the stent graft 106, as described below.

Referring back to FIGS. 1A-1D, and as previously described, the radiopaque stent graft 100 may include a functionalized surface 107, namely, a surface that has been subjected to a surface modification. Referring again to FIGS. 2-3, In embodiments, the surface modification may include plasma treatment of the exterior surface of the vascular graft 105 of the base stent graft 101. In embodiments, an exterior surface of the vascular graft 105 is subjected to an exemplary plasma treatment process to functionalize the exterior surface of the vascular graft 105 and thereby produce a functionalized surface 107. As stated previously, in embodiments, the vascular graft 105 may be formed of a polymer material. In exemplary embodiments, the vascular graft 105 may be formed of a polymer material that comprises polytetrafluoroethylene (PTFE), polyvinyl chloride (PVC), polyethylene terephthalate (PET), polyethylene, Nylon, PEBAX (i.e. a copolymer of polyether and polyamide), polystyrene (PS), polyethleneterephthalate (PETP), or various other suitable materials as will be apparent to those of ordinary skill in the art. The plasma treatment process may increase the functionality of the exterior surface of the vascular graft 105, thereby producing functionalized surface 107 so that one or more layers may be applied to the stent graft 106.

Referring still to FIGS. 2-3, In embodiments, the plasma treatment process is configured to functionalize the base stent graft 101 along the exterior surface of the vascular graft 105 and/or other portions of the of the vascular graft 105. By functionalizing the exterior surface of the vascular graft 105 prior to the application of the adherent layer 115 (as shown in FIG. 4 described subsequently) onto the base stent graft 101, the plasma treatment process is operable to modify the material properties of the exterior surface of the vascular graft 105 to thereby provide improved interactions between the adherent layer 115 and the functionalized surface 107. For example, treating the exterior surface of the vascular graft 105 with the plasma treatment process allows for the positioning of one or more radiopaque markers onto the stent graft 106 (via the adherent layer) without damaging the stent graft 106.

Referring still to FIGS. 2-3, In exemplary use, at least a portion of the base stent graft 101 is positioned within a plasma chamber (not shown) for treating pursuant to the plasma treatment process. In the present example, the base stent graft 101 is positioned within the plasma chamber such that exterior surface of the vascular graft 105 is treated by the plasma treatment process. Although the treatment of the exterior surface of the vascular graft 105 is described in the present embodiments, it should be understood that other various portions of the base stent graft 101 and/or other suitable implantable prostheses may be treated with plasma treatment process of the present disclosure as will be apparent to those of ordinary skill in the art. With the base stent graft 101 positioned therein, the plasma treatment process commences by generating a desired vacuum within the chamber. In this instance, the exterior surface of the vascular graft 105 is subjected to the vacuum atmosphere generated within the chamber. During the plasma treatment, the temperatures and pressures used within the chamber may depend on the materials of the base stent graft 101. For example, too high of a temperature may destroy materials of the base stent graft 101. In embodiments, the temperature within the chamber may be from 25° C. to 50° C., from 20° C. to 40° C., from 20° C. to 30° C., from 30° C. to 50° C., from 30° C. to 40° C., or from 40° C. to 50° C.

With the vacuum pressure maintained within the chamber, the polymer material of the exterior surface of the vascular graft 105 is exposed to a gas supplied by a gas source (not shown) coupled to the plasma chamber. In embodiments, the exterior surface of the vascular graft 105 may be exposed to xenon, argon, fluorine, a fluorocarbon gas mixture, a xenon/oxygen gas mixture, or other suitable gas mixture supplied by the gas source. In specific embodiments, the exterior surface of the vascular graft 105 may be exposed to xenon, argon, or a xenon/oxygen gas mixture supplied by the gas source. Depending on the type of gas supplied, the gas source is operable to generate ions (i.e. fluorinated ions) and radicals from the gas transmitted into the chamber. In some embodiments, the gas source is operable to generate fluorine-containing ions (i.e. fluorinated ions) and radicals from the fluorine or fluorocarbon gas transmitted into the chamber. By way of example only, the gases that may be transmitted by the gas source may comprise tetrafluoromethane (CF₄), hexafluoroethane (C₂F₆), xenon difluoride (XeF₂), fluorine (F₂), chloropentafluoroethane (CF₃CCIF₂), sulfur hexafluoride (SF₆), and other suitable gases as will be apparent to those of ordinary skill in the art. The discharge of the mixture into the chamber subjects the exterior surface of the vascular graft 105 to the gas at a predetermined flow and for a predetermined duration.

In embodiments, then, a plasma generator (not shown) that is in fluidic communication with the chamber is activated to thereby discharge plasma into the container and thereby expose the exterior surface of the vascular graft 105 to the plasma. As merely an illustrative example, the plasma generator may comprise an arc discharger, a dielectric barrier discharger, a spark discharger, resistive barrier dischargers, radio-frequency excitation, microwave frequency excitation, and other suitable generators as will be apparent to those of ordinary skill in the art.

The exterior surface of the vascular graft 105 may be treated with the plasma sourced from the plasma generator such that the plasma interacts with the polymer material of the exterior surface of the vascular graft 105 at a predetermined flow. In embodiments, the generated plasma comprises ions and radicals. The plasma ion-implantation step may comprise initially extracting ions from the plasma generator and subsequently selecting desired ions for transmission through an accelerating column via a magnetic field (not shown) of the plasma generator. In this instance, the ions selected by the magnetic field are forced into the polymer material of the exterior surface of the vascular graft 105 such that the ions are effectively implanted into the of the vascular graft 105, thereby producing the functionalized surface 107. Hydrogen atoms of the polymer material of the exterior surface of the vascular graft 105 are simultaneously replaced by the ion species implanted thereon.

In an exemplary embodiment, the exterior surface of the vascular graft 105 may be fluorinated with the plasma sourced from the plasma generator such that the plasma interacts with the polymer material of the exterior surface of the vascular graft 105 at a predetermined flow. In the present example, the generated plasma comprises fluorine-containing ions and radicals. The plasma ion-implantation step may comprise initially extracting fluorine-containing ions from the plasma generator and subsequently selecting desired ions for transmission through an accelerating column via a magnetic field (not shown) of the plasma generator. In this instance, the fluorine-containing ions selected by the magnetic field are forced into the polymer material of the exterior surface of the vascular graft 105 such that the ions are effectively implanted into the exterior surface of the vascular graft 105. Hydrogen atoms of the polymer material of the exterior surface of the vascular graft 105 are simultaneously replaced by the fluorinated species implanted thereon.

In embodiments, then, the portion of the base stent graft 101 contained within the plasma chamber (i.e. the exterior surface of the vascular graft 105) is continuously treated with the plasma for a predetermined duration, thereby forming a functionalized surface 107 along the stent graft 106. The plasma treatment process may be concluded once the exterior surface of the vascular graft 105 has been exposed to the plasma for the predetermined duration, resulting in the functionalized surface 107.

Referring to FIG. 3, in embodiments, after producing the functionalized surface 107 a tie layer 110 may be disposed over the functionalized surface 107. The term “tie layer” as used herein, refers to a layer that adheres two layers together. For example a tie layer may bond polar materials to one or more layers that do not include polar materials. In embodiments, the tie layer may include any material capable of adhering the functionalized surface 107 to the subsequently applied adherent layer 115, which is described in more detail below. In some embodiments, the tie layer 110 may be a silane layer.

In embodiments, application of the tie layer 110 may be applied by various methods known in the art. For example, the tie layer 110 may be coated onto functionalized surface 107 by various coating methods known in the art, for example, by dipping, spraying, inkjet printing, other methods, or combinations. In other embodiments, the tie layer 110 may be applied onto functionalized surface 107 by a plasma treatment process. The plasma treatment process may be selected based on the materials of the tie layer 110. In some embodiments, the plasma treatment method applying the tie layer 110 onto functionalized surface 107 may not utilize an electric field within the plasma chamber. In embodiments, the tie layer 110 may be applied onto functionalized surface 107 by a plasma treatment process, which is performed in the same plasma chamber as previously described. Alternatively or additionally, the tie layer 110 may be applied onto functionalized surface 107 outside of the plasma chamber previously described or in a different plasma chamber. Embodiments of applying the tie layer 110 onto functionalized surface 107 may further include combinations of coating methods and plasma treatment methods.

As seen in FIG. 3, the tie layer 110 disposed onto the functionalized surface 107 may be relatively minimal and include a substantially uniform distribution along the surface area of the functionalized surface 107. By way of example only, the thickness of tie layer 110 implanted onto the functionalized surface 107 is less than one micron. In embodiments, the tie layer 110 may be from 10 angstroms (Å) to 1000 Å, from 10 Å to 750 Å, from 10 Å to 500 Å, from 10 Å to 250 Å, from 10 Å to 100 Å, from 100 Å to 1000 Å, from 100 Å to 750 Å, from 100 Å to 500 Å, from 100 Å to 250 Å, from 250 Å to 1000 Å, from 250 Å to 750 Å, from 250 Å to 500 Å, from 500 Å to 1000 Å, from 500 Å to 750 Å, or from 750 Å to 1000 Å.

With the plasma treatment process producing the functionalized surface 107 being complete and the tie layer 110 disposed thereon being complete; the treatment process effectively forms a surface upon which an adherent layer may be disposed. As should be understood in view of the teachings herein, optimal results may be achieved during the plasma treatment process by applying appropriate gas types, gas flow, treatment times, and generator power.

With the minimal thickness of the tie layer 110 disposed onto the functionalized surface 107, an adherent layer 115 is disposed onto the tie layer 110, as seen in FIG. 4. In embodiments, the adherent layer 115 allows for the positioning of one or more radiopaque markers onto the stent graft 106 (via the adherent layer) without damaging the vascular graft of the stent graft 106.

The adherent layer 115 may include a polymer. Particularly suitable polymers of the adherent layer 115 include biocompatible polymers that avoid undesirable irritation of body tissue. Example polymers include polymers formed from cycloaliphatic monomers or aromatic monomers. Examples of cycloaliphatic monomers include alkylcyclohexanes such as methylcyclohexane. Examples of aromatic monomers include alkylbenzenes such as toluene and xylenes. In some embodiments, the intermediate layer may be a poly(p-xylylene) such as parylene C, parylene N, parylene D, parylene X, parylene AF-4, parylene SF, parylene HT, parylene VT-4 (parylene F), parylene CF, parylene A, or parylene AM, for example. Structures of selected parylenes are provided below:

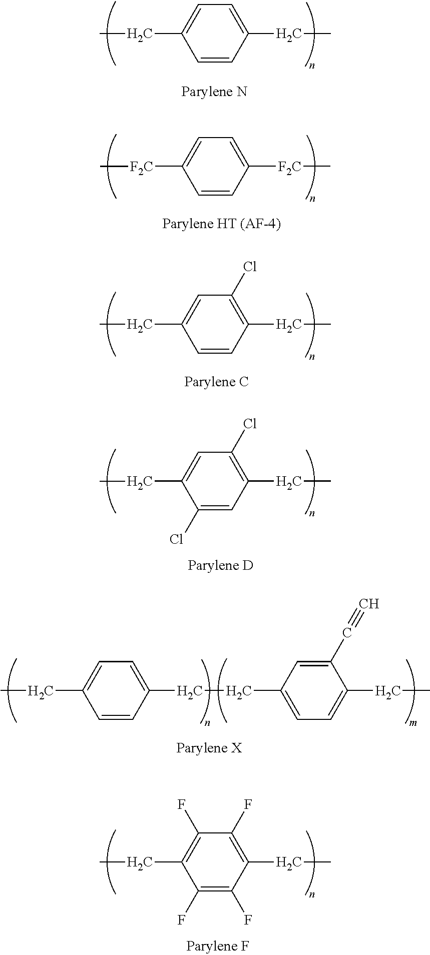

Additional polymers may be present in the adherent layer 115. Examples of such additional polymers include, for example, polyolefins, polyisobutylene, ethylene-α-olefin copolymers, acrylic polymers and copolymers, polyvinyl chloride, polyvinyl methyl ether, polyvinylidene fluoride and polyvinylidene chloride, polyacrylonitrile, polyvinyl ketones, polystyrene, polyvinyl acetate, ethylene-methyl methacrylate copolymers, acrylonitrile-styrene copolymers, ABS resins, Nylon 12 and its block copolymers, polycaprolactone, polyoxymethylenes, polyethers, epoxy resins, polyurethanes, rayon-triacetate, cellulose, cellulose acetate, cellulose butyrate, cellophane, cellulose nitrate, cellulose propionate, cellulose ethers, carboxymethyl cellulose, chitins, polylactic acid, polyglycolic acid, polylactic acid-polyethylene oxide copolymers, polyethylene glycol, polypropylene glycol, polyvinyl alcohol, and mixtures and block copolymers thereof. In embodiments, the additional polymers may be chosen from polymers having a low surface free-energy.

Without intent to be bound by theory, it is believed that adherent layers including certain polymer materials such as parylenes, for example, may adhere to the functionalized surface 107 via the tie layer 110) and thereby produce a layer upon which one or more radiopaque markers may be applied to the stent graft 106.

Because the implantable prostheses according to embodiments, particularly stent grafts, for example, undergo mechanical manipulation, i.e., expansion and contraction, further examples of polymers that may be useful in the adherent layer 115 may include elastomeric polymers, such as silicones (e.g., polysiloxanes and substituted polysiloxanes), polyurethanes, thermoplastic elastomers, ethylene vinyl acetate copolymers, polyolefin elastomers, and EPDM rubbers. Because of the elastic characteristics of these polymers, when these polymers are included in the adherent layer 115, adherence of the radiopaque markers to the surface of the adherent layer 115 and ultimately to the implantable prostheses may increase when the implantable prostheses is subjected to forces or stress.

The adherent layer 115 may comprise one or more other components in order to maintain the integrity and adherence of the one or more radiopaque markers and/or sealant layers to the implantable prostheses, to facilitate both adherence of radiopaque markers and additive components during transit and deployment at the treatment site within the body vessel, or combinations of these benefits.

In embodiments, the adherent layer 115 may have a thickness from 1 microns to 5 microns, from 1 microns to 4 microns, from 1 microns to 3 microns, from 1 microns to 2 microns, from 2 microns to 5 microns, from 2 microns to 4 microns, from 2 microns to 3 microns, from 3 microns to 5 microns, from 3 microns to 4 microns, or from 4 microns to 5 microns.

Referring now to FIG. 5, in embodiments, one or more slots may be etched into the adherent layer 115 thereby producing an etched layer 117. As used herein, “etching” means creating a surface modification in the surface of the adherent layer 115. The etching methods may be used to etch one or more slots 119 into the adherent layer 115 to produce the etched layer 117. The one or more slots 119 may allow for improved adherence of the radiopaque markers within the adherent layer 115.

Various etching methods are contemplated, which may include utilizing chemical methods, mechanical methods, lasers, other etching methods, and combinations thereof. In embodiments, etching may include utilizing solvents to selectively dissolve the surface of the adherent layer 115. In embodiments, etching may include utilizing a laser. In further embodiments, the laser may be an excimer laser. In embodiments, etching may include utilizing a mechanical etching device, such as blades or shears. Selection of a particular etching method may depend on desired efficiency of the overall process and/or the desired precision of the method. In a specific embodiment, an excimer laser etching may be selected to produce one more slots with precise dimensions.

The size and number of the one or more slots 119 may depend on the desired radiopacity of the radiopaque stent graft 100. In some embodiments, only one slot may be desired. In other embodiments, one or more additional slots may be desired. In embodiments, the one or more slots may be etched in any orientation or in any pattern along the surface of the adherent layer 115. For example, in embodiments, the one or more slots 119 may be etched in radial directions, longitudinal directions, or both.

Referring still to FIG. 5, in embodiments, the etched layer 117 may be subjected to a second plasma treatment process. The second plasma treatment process may allow for improved adherence of the one or more radiopaque markers onto the etched layer 117 (via the one or more slots 119). As previously explained, the etched layer 117 is formed of a polymer. Applying the second plasma treatment process to the polymer material of the etched layer 117 may functionalize the etched layer 117.

In the present example, the second treatment process may be configured to improve the adherence properties of the etched layer 117. By functionalizing the surface of the etched layer 117 prior to the application of one or more radiopaque markers into the one or more slots 119, the second plasma treatment process may modify the material properties of the etched layer 117 to thereby provide improved interactions between the one or more radiopaque markers and the etched layer 117. For example, treating the surface of the etched layer 117 with the second plasma treatment process may allow for the one or more radiopaque markers to adhere to the etched layer 117. Ultimately, the second plasma treatment process may be operable to provide a radiopaque stent graft 100 by a method that prevents damage to the stent graft 106.

In exemplary use, at least a portion of the etched layer 117 may be positioned within a second plasma chamber (not shown) for treating pursuant to the second plasma treatment process. In the present example, at least a portion of the etched layer 117 may be positioned within the plasma chamber such that the etched layer 117 is treated by the second plasma treatment process. With at least a portion of the etched layer 117 positioned therein, the second treatment process may commence by generating a desired vacuum within the second plasma chamber. In this instance, the etched layer 117 may be subjected to the vacuum atmosphere generated within the chamber. The temperature within the chamber may depend on the materials of the etched layer 117. In embodiments, the temperature within the chamber may be from 25° C. to 50° C., from 20° C. to 40° C., from 20° C. to 30° C., from 30° C. to 50° C., from 30° C. to 40° C., or from 40° C. to 50° C.

With the vacuum pressure maintained within the chamber, the polymer material of the exterior surface of the vascular graft 105 is exposed a gas supplied by a gas source (not shown) coupled to the plasma chamber. In embodiments, the etched layer 117 may be exposed to xenon, argon, fluorine, a fluorocarbon gas mixture, a xenon/oxygen gas mixture, an acrylate gas or other suitable gas mixture supplied by the gas source. In specific embodiments, the etched layer 117 may be exposed to a xenon and acrylate gas mixture supplied by the gas source. Depending on the type of gas supplied, the gas source is operable to ions (i.e. fluorinated ions) and radicals from the gas transmitted into the chamber. In some embodiments, the gas source is operable to generate fluorine-containing ions (i.e. fluorinated ions) and radicals from the fluorine or fluorocarbon gas transmitted into the chamber. By way of example only, the gases that may be transmitted by the gas source may comprise tetrafluoromethane (CF₄), hexafluoroethane (C₂F₆), xenon difluoride (XeF₂), fluorine (F₂), chloropentafluoroethane (CF₃CCIF₂), sulfur hexafluoride (SF₆), and other suitable gases as will be apparent to those of ordinary skill in the art. The discharge of the mixture into the chamber subjects the etched layer 117 to the gas at a predetermined flow and for a predetermined duration.

In embodiments, then, a second plasma generator (not shown) that is in fluidic communication with the chamber may be activated to thereby discharge plasma into the container and thereby expose the etched layer 117 to the plasma. As merely an illustrative example, the second plasma generator may comprise an arc discharger, a dielectric barrier discharger, a spark discharger, resistive barrier dischargers, radio-frequency excitation, microwave frequency excitation, and other suitable generators as will be apparent to those of ordinary skill in the art.

The etched layer 117 may be fluorinated with the plasma sourced from the second plasma generator such that the plasma interacts with the polymer material of the etched layer 117 at a predetermined flow. In the present example, the generated plasma comprises ions and radicals. The plasma ion-implantation step may comprise initially extracting ions from the second plasma generator and subsequently selecting desired ions for transmission through an accelerating column via a magnetic field (not shown) of the second plasma generator. In this instance, the ions selected by the magnetic field are forced into the polymer material of the etched layer 117 such that the ions are effectively implanted into the etched layer 117. Hydrogen atoms of the polymer material of the etched layer 117 are simultaneously replaced by the ion species implanted thereon.

In embodiments, then, the portion of the etched layer 117 contained within the second plasma chamber may be continuously treated with the plasma for a predetermined duration, thereby forming a functionalized surface along the etched layer 117. The second plasma treatment process may be concluded once the etched layer 117 has been exposed to the plasma for a predetermined duration, resulting in the etched layer 117 comprising a plasma layer (not shown) implanted thereon, thereby functionalizing the etched layer 117.

In embodiments, a functionalized plasma layer (not shown) may be implanted onto the etched layer 117, which may be relatively minimal and may include a substantially uniform distribution along the surface area of the etched layer 117. By way of example only, the thickness of plasma layer implanted onto the etched layer 117 may be less than one micron. With the reaction of the polymer surface of the etched layer 117 and plasma layer applied thereon being complete; the second plasma treatment process effectively forms a functionalized surface upon which the one or more radiopaque markers may be applied with improved adherence. As should be understood in view of the teachings herein, optimal results may be achieved during the second plasma treatment process by applying appropriate gas types, gas flow, treatment times, and generator power. With the minimal thickness of the plasma layer applied onto the etched layer 117, the one or more radiopaque markers may be applied into one or more slots 119 of the etched layer 117.

Referring now to FIG. 6, in embodiments, a radiopaque marker may be positioned in each of the one or more slots 119, producing filled slots 120, and thereby producing a radiopaque stent graft 100. The radiopaque markers may fill at least a portion of the one or more slots 119 in the etched layer 117. Accordingly, the one or more filled slots 120 comprising one or more radiopaque markers allow for fluoroscopic visualization of the radiopaque stent graft 100 during implantation. In embodiments, the one or more radiopaque markers have radiopaque features incorporated therein, which can be used as visual markers located by fluoroscopy.

The radiopaque markers may comprise a radiopaque material. For example, radiopaque materials may include polymers, gold, platinum-iridium alloy, bismuth subcarbonate, barium sulfate, bismuth oxychloride, bismuth trioxide, tungsten, combinations, and various other radiopaque materials know to those skilled in the art.

Referring now to FIG. 7, in embodiments of the radiopaque stent graft 100, the etched layer 117 having filled slots 120 may be optionally coated with an adhesive, producing a sealant layer 125. The sealant layer 125 may encapsulate the one or more radiopaque markers within the one or more filled slots 120 to seal the one or more radiopaque markers within the one or more filled slots 120 of the etched layer 117. In embodiments, the sealant layer 125 may fully encapsulate the one or more radiopaque markers within the one or more filled slots 120 to prevent the one or more radiopaque markers from being exposed to the surrounding environment for safety.

The sealant layer 125 may comprise an adhesive that has been coated onto the etched layer 117 having filled slots 120. The adhesive utilized may be selected based on the radiopaque material selected above and other properties, including biocompatibility. In embodiments, adhesives used to produce the sealant layer 125 may include flexible acrylate adhesives.

The adhesive may be coated onto the etched layer 117 having filled slots 120 by various coating methods known in the art. In embodiments, the adhesive may be coated onto the etched layer 117 having filled slots 120 by dipping, spraying, or other methods. In embodiments, the method may further comprise curing the adhesive to form the sealant layer 125. In embodiments, curing the adhesive may including exposing the adhesive to a ultraviolet light emitted by an ultraviolet light source (not shown). In embodiments, the adhesive may be exposed to the ultraviolet light at an intensity and for a duration sufficient to completely cure the adhesive and thereby produce the sealant layer 125. In further embodiments, one or more additional layers may be applied, for example, to improve the biocompatibility of the radiopaque stent graft 100.

Treatment Methods Using the Radiopaque Stent Grafts

Embodiments will now be described for treatment methods that utilizing the radiopaque stent grafts described herein.

In embodiments, the radiopaque stent graft 100 may be delivered via a small incision on a mammalian body. In embodiments, the radiopaque stent graft 100 may be delivered to a damaged or diseased site (the treatment site) via a constraining member. Constraining members may include a catheter or sheath. In embodiments, the constraining member and the radiopaque stent graft 100 may together be inserted into the small incision, guided through the body vessel to the treatment site, and deployed by removing the constraining member. In embodiments, the radiopaque stent graft 100 may then self-expand at normal body temperature once placed at the treatment site and after the constraining member has been removed.

To direct the radiopaque stent graft 100 to the precise location of the treatment site, the radiopaque markers of the radiopaque stent graft 100 may be visualized using a fluoroscope by means of X-ray. In embodiments, the radiopaque stent graft 100 may be used to confirm the correct placement within the body vessel. The radiopaque stent graft 100 may be used to visually verify the orientation of the radiopaque stent graft 100 to determine whether the radiopaque stent graft 100 has been mislocated, twisted, or kinked. Utilizing the radiopaque stent graft 100 described herein may reduce the risk of deployment of the radiopaque stent graft 100 at an unintended location, which could result in immediate trauma. Utilizing the radiopaque stent graft 100 described herein may reduce the risk of increased invasiveness associated with multiple deployment attempts and relocation of the constraining member.

It should now be understood that embodiments as described herein are directed the systems, methods, and catheters for treatment of a body vessel. In particular, embodiments as described herein include radiopaque stent grafts that may allow for visualization and therefore provide treatment methods that are minimally invasive.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method of producing a radiopaque stent graft comprising:
treating an exterior surface of a base stent graft to produce a functionalized surface;
disposing a tie layer over the functionalized surface;
disposing an adherent layer over the tie layer;
etching a slot into the adherent layer to produce an etched layer;
treating the etched layer; and
positioning a radiopaque marker in the slot of the etched layer to produce a filled slot and thereby produce the radiopaque stent graft.

2. The method of claim 1, wherein the base stent graft comprises polytetrafluoroethylene.

3. The method of claim 1, wherein the adherent layer comprises a poly(p-xylylene).

4. The method of claim 3, wherein the poly(p-xylylene) comprises one or more of parylene C, parylene N, parylene D, parylene X, parylene AF-4, parylene SF, parylene HT, parylene VT-4 (parylene F), parylene CF, parylene A, or parylene AM.

5. The method of any preceding claim 1, further comprising coating the filled slot with an adhesive to produce a sealant layer.

6. The method of claim 1, further comprising:
etching one or more additional slots in to the adherent layer;
plasma-treating the one or more additional slots;
positioning a radiopaque marker into each of the one or more additional slots to produce one or more additional filled slots and thereby produce the radiopaque stent graft.

7. The method of claim 6, further comprising coating the filled slots with an adhesive to produce a sealant layer.

8. The method of claim 7, wherein coating the one or more additional filled slots comprises utilizing a flexible acrylate adhesive.

9. The method of claim 8, further comprising curing the adhesive to produce the sealant layer.

10. The method of claim 1, wherein etching comprises laser-etching.

11. The method of claim 1, wherein treating the exterior surface of a base stent graft comprises plasma-treating the exterior surface.

12. The method of claim 1, wherein treating the etched layer comprises plasma-treating the etched layer.

13. A radiopaque stent graft comprising:
a stent graft having a functionalized surface;
a tie layer surrounding a functionalized surface;
an etched layer covering at least a portion of the tie layer, wherein the etched adherent layer comprises a filled slot, and wherein the filled slot comprises a radiopaque marker.

14. The radiopaque stent graft of claim 13, further comprising a sealant layer encapsulating the filled slot.

15. The radiopaque stent graft of claim 13, wherein the sealant layer comprises a flexible acrylate adhesive.

16. The radiopaque stent graft of claim 13, wherein the base stent graft comprises polytetrafluoroethylene.

17. The radiopaque stent graft of claim 13, wherein the etched layer further comprises a poly(p-xylylene).

18. The radiopaque stent graft of claim 17, wherein the poly(p-xylylene) comprises one or more of parylene C, parylene N, parylene D, parylene X, parylene AF-4, parylene SF, parylene HT, parylene VT-4 (parylene F), parylene CF, parylene A, or parylene AM.

19. The radiopaque stent graft of claim 13, further comprising one or more additional filled slots, wherein the one or more additional filled slots each comprise a radiopaque marker.

20. (canceled)

21. The radiopaque stent graft of claim 13, wherein the etched layer is functionalized to securely affix the radiopaque marker to the slot.

22. (canceled)
